(19) United States
(12) Patent Application Publication
(10) Pub. No.: US 2024/0278187 A1
(43) Pub. Date: Aug. 22, 2024

(54) PLANT-DERIVED VESICLES INCORPORATING TRANS-MEMBRANE PROTEINS

(71) Applicant: AQUAPORIN A/S, Kongens Lyngby (DK)

(72) Inventors: Reyhan SENGUR-TASDEMIR, Holte (DK); Torsten Høybye Bak REGUEIRA, Værløse (DK)

(73) Assignee: AQUAPORIN A/S, Kongens Lyngby (DK)

(21) Appl. No.: 18/566,426

(22) PCT Filed: Jun. 3, 2022
(86) PCT No.: PCT/EP2022/065245

§ 371 Date: Dec. 1, 2023

(30) Foreign Application Priority Data
Jun. 3, 2021 (DK) PA202170289

(51) Int. Cl. B01D 69/14; A61K 9/127; A61K 36/21; B01D 63/08; B01D 69/10; B01D 69/12; B01D 71/56

(52) U.S. Cl. CPC B01D 69/144; A61K 9/1272; A61K 9/1277; A61K 36/21; B01D 63/08; B01D 69/10; B01D 69/1251; B01D 71/56

(57) ABSTRACT

The disclosure relates to a vesicle in a liquid composition, the vesicle including plant-derived transmembrane proteins. The disclosed embodiments further relate to methods of producing the vesicles and separation membranes of such vesicles. The present disclosure provides for the preparation of a separation membrane using vesicle components originating exclusively or mainly from natural sources.

### TECHNICAL FIELD

The disclosure relates to a vesicle in a liquid composition comprising plant-derived transmembrane proteins. The present invention further relates to methods of producing the vesicles, separation membranes comprising of such vesicles, and a method of preparing a thin film composite layer immobilizing the vesicles.

### BACKGROUND

The use of amphiphilic lipids and block copolymers for forming self-assembled vesicles having bilayer or bilayer-like structures is well known in the art, in particular for immobilizing amphiphilic membrane proteins, such as aquaporin vesicle water channels. Synthetic structures, called polymersomes and comprising aquaporin water channels, can then be used to make membranes having immobilised aquaporin water channels for applications such as the purification of water (WO2006/122566) or the generation of salinity power (WO2007/033675), in general by depositing the vesicles as a layer or in a film on a supporting substrate, which allows the selective passage of water molecules through the membranes by nanofiltration, reverse osmosis, forward osmosis or pressure retarded osmosis.

WO20014483 discloses compositions and methods for producing plant plasma membranes supported on a surface of an object (e.g. on-a-chip). WO20182938 discloses plant-derived extracellular vesicle compositions and their uses. Annette Lund and Anja hoe Fuglsang discloses in Methods in Molecular Biology, vol. 913, p. 217, DOI 10.1007/978-1-61779-986-0_14 the purification of plant plasma membranes by two-phase partitioning and measurement of H+ pumping. The two-phase was based on the separation of microsomal membranes, dependent on their surface hydrophobicity.

Typically, in the prior art, the plant-derived vesicles are not employed for separation processes due to the high-pressure environment or high saline concentrations requiring materials with high stability. Plant-derived vesicles may rupture under high pressure or high saline conditions and is an object of the present invention to stabilise the vesicles so that they can be use in industrial membrane processes.

Typically, transmembrane proteins, such as aquaporin proteins, are embedded in said vesicles into the polyamide layer during coating of selective membranes. The aquaporins are first incorporated into polymersomes by a formulation process. In the formulation process several surfactants, chemicals and polymers are present to secure the formation of stable polymersomes. Polymersome preparation is performed via bulk hydration self-assembly of poly (dimethyloxazoline)-poly (dimethylsiloxane)-poly (dimethyloxazoline) (PMOXA-PDMS-PMOXA) blockcopolymerandpoly (dimethyloxazoline)-poly (dimethylsiloxane) (PMOXA-PDMS) block copolymer blend that was combined with reactive end group functionalized PDMS, such as NH2 terminated PDMS (A-PDMS). Once the polymersomes are self-assembled and include the AQP protein produced in E. coli it is then included in the polyamide layer. Also, before the transmembrane protein is mixed with the other constituents, the transmembrane protein is typically solubilized in a detergent. The solubilization of the transmembrane protein in a detergent prevents or ameliorates the tendency of the transmembrane protein to precipitate in the aqueous solution.

After formulation, the aquaporin containing polymersomes are added to the water phase and during coating it is incorporated into the selective polyamide layer.

The amphiphilic lipids and block copolymers used in vesicle production are solids that need to be dissolved in harsh solvents, such as tetrachloromethane (CCl4) or chloroform (CHCl3), to solubilise their predominantly hydrophobic portions. During membrane synthesis, this solvent is evaporated to allow film formation. This harsh solvent evaporation is detrimental to the environment when carried out on an industrial scale.

Following film formation by evaporation of solvents, the film is rehydrated to bring the amphiphile into various emulsion forms (such as vesicles), with simultaneous incorporation of the AQP membrane protein. However, in practice, it is often difficult to control the t final vesicle size, resulting in disperse emulsions having vesicles ranging in diameter of from about 60 to 80 nm to about 1000 nm or more. There are also limits to the number of AQPs that can be incorporated in each vesicle, because the membrane proteins need to be aligned according to their amphiphilic structure in the bilayer structure and to match the thickness of the hydrophobic part of the protein and vesicle membrane.

The foregoing and other objects are achieved by the features of the independent claims. Further implementation forms are apparent from the dependent claims, the description and the figures.

### SUMMARY

It is a first object to provide a vesicle that comprises an ideal number of transmembrane proteins in a liquid composition. It is another object of the present disclosure to provide a vesicle that binds efficiently to a polyamide layer on a selective membrane that can achieve an acceptable membrane performance. It is yet a further object of the present disclosure to provide a food contact approved selective membrane incorporating food contact approved water channels. It is yet a further object of the present disclosure to provide a method for preparing vesicles for use in a selective membrane in a liquid composition that avoids the use of detergents, surfactants and/or harsh solvents, which are detrimental to the environment.

Thus, in a first aspect there is provided vesicle in a liquid composition, said vesicle comprising plant-derived transmembrane proteins, wherein the vesicle-forming material comprises plant plasma membrane components.

It has surprisingly been shown by the present inventors that is possible to the obtain a vesicle comprising plant plasma membrane components and incorporating a plant-derived transmembrane protein in the membrane of the vesicle. The vesicle may be used as such in the production of separation membranes without incorporating substantial amounts of synthetic components. Thus, the present disclosure provides for the preparation of a separation membrane using vesicle components originating exclusively or mainly from natural sources. The separation membrane may be used for the preparation of membrane modules useful for household water purifiers.

According to a possible implementation form of the first aspect, the plant plasma membrane components originate from the same plant as the plant-derived transmembrane proteins. By the plasma membrane components being derived from the same plant as the plant-derived transmembrane proteins, the compatibility of transmembrane proteins in a lipid bilayer may be increased. Thus, a vesicle that comprises an increased number of transmembrane proteins in a liquid composition may be obtained; without wanting to be bound by any particular theory, it is believed that by using transmembrane proteins and vesicle membranes that are derived from a same plant the loss of transmembrane protein incorporation due to requirement of transmembrane proteins to match the thickness of the hydrophobic part of the protein to the membrane thickness and transmembrane protein misalignment is avoided.

In a possible implementation form of the first aspect the vesicle forming material consists of plant plasma membrane components. By the vesicles being obtained from a plant source while including naturally occurring transmembrane proteins and plasma membrane components, non-synthetic lipid bilayer vesicles incorporating ion transport (ion channels) and/or water channels (aquaporin water channels) may be obtained. Without wanting to be bound by any particular theory, it is believed that by using vesicles obtained from a plant source and naturally including transmembrane proteins and plasma membrane components, the loss of transmembrane protein incorporation due to requirement of transmembrane proteins to match the thickness of the hydrophobic part of the protein to the membrane thickness and transmembrane protein misalignment is avoided. Thereby, greater reproducibility of the performance of the selective layer, as measured, e.g., by flux (LMH/Bar) and salt rejection (%), may be ensured when comparing to the reproducibility of performance of polymersome produced selective membranes. This effect may be most apparent in industrial scale membrane production.

Moreover, when said transmembrane protein comprises an ion channel or an aquaporin or the like and said vesicles comprising said transmembrane protein are immobilized or incorporated in an active or selective layer, the transmembrane protein maintains its biologically active folded structure when complexed in the vesicle membrane wherein it may be shielded from degradation at each step of the vesicle isolation process. Thereby, the transmembrane proteins may remain sufficiently stable without the addition of further components to the liquid composition, and, thus, may preserve their desired functionality when processed into separation membranes in lab and industrial scale, while offering increased integration of viable vesicles into the selective membrane active layer. Also, the use of harsh detergents that may be detrimental to the environment is avoided.

Further, by the liquid composition only including plant-derived vesicles, a food contact approved selective membrane may be obtained.

Yet further, it was found that plant-derived vesicles may be stabilized onto the support membrane so as to withstand the necessary pressure and other environmental conditions when in use.

Thus, while the vesicle forming materials and the plant derived transmembrane proteins may be obtained from different sources, it is generally desirable that they originate from the same plant, or are derived without isolating the protein from the plasma membrane. Plant plasma membrane components and plant derived transmembrane proteins may, alternatively, be obtained from different the plant sorts.

In a possible implementation form of the first aspect the plant-derived transmembrane protein is an aquaporin water channel. While any transmembrane protein may by incorporate in the membrane material disclosed in the present invention, it is generally desired to use transmembrane protein that transport ions (ion channels) or water (aquaporin water channels). Ion channels include chloride channels and metal ion transporters. Chloride channels in addition to the chloride ion also conducts HCO₃⁻, I⁻, SCN⁻, and NO₃⁻ in some transmembrane proteins. The metal ion transporters include magnesium transporters, potassium ion channels, sodium ion channels, calcium channels, proton channels etc.

Aquaporin water channels are transmembrane proteins widely occurring in nature for selective transportation of water in or out of cells. In an industrial setting, the aquaporin water channels in a semi-permeable membrane ensure the flow of water by osmosis, while other solutes in the solution are rejected. The presence of active aquaporin water channels thus assists the semi-permeable membrane rejecting solutes and in promoting the penetration of water through the membrane. In a possible implementation form of the first aspect the liquid composition comprises an aqueous buffer.

In a possible implementation form of the first aspect, the aqueous buffer acidity is equal to or above pH 7.0, such as equal to or above pH 7.5, such as equal to or above pH 7.8, such as equal to or above pH 8. By the aqueous buffer being slightly basic, the stability of the vesicles in the liquid composition may be enhanced.

In a possible implementation form of the first aspect the liquid composition comprises a reactive end group functionalised PDMS (polydimethylsilane).

In a possible implementation form of the first aspect the reactive end group functionalised PDMS is functionalized with one or more of amine, carboxylic acid, and/or hydroxy group (s), and wherein the number of monomers is in the range of 30 to 50.

In a possible implementation form of the first aspect the reactive end group functionalised PDMS is poly(dimethylsiloxane), bis(3-aminopropyl).

By the liquid composition comprising a reactive end group functionalised PDMS, at least some of this functionalized PDMS may intergrate into the vesicle membrane before coating on the selective layer. Without wishing to be bound by any particular theory, it is believed that the vesicles containing free available reactive groups on the surface will be not only physically incorporated or immobilised (adsorbed), but also chemically bound in a TFC layer, because the reactive free end groups, such as amino groups, hydroxyl groups and carboxyl groups, will participate in the interfacial polymerization reaction with the acyl chloride, such as a trimesoyl chloride (TMC). In this way, it is believed that vesicles will be covalently bound in the TFC layer, leading to relatively higher vesicle loading and thus higher water flux through the membranes. In addition, it is believed that the covalent coupling of vesicles in the TFC layer results in higher stability and/or longevity of the AQPs and AQP-vesicles when incorporated in the selective membrane layer.

In a possible implementation form of the first aspect the plant-derived components comprise phospholipids.

In a possible implementation form of the first aspect the liquid composition comprises a flux increasing agent.

In a possible implementation form of the first aspect the flux improving agent is an alkylene glycol monoalkyl ether alkylat, beta cyclodextrin, polyethylene glycol-hydroxystearate, or a combination thereof.

In a possible implementation form of the first aspect the flux increasing agent is polyethylene glycol-hydroxystearate.

In a possible implementation form of the first aspect the liquid composition comprises a detergent or a surfactant.

Since, in a different possible implementation form of the first aspect, the transmembrane proteins of the present disclosure are not solubilized but, rather, are included in the plasma membrane components derived from plant source, there is no need for detergent or surfactant to be present in the liquid composition for the purpose of increasing protein solubility. This has the advantage of reducing the number of components required for obtaining a formulation that can lead to sufficient loading of transmembrane proteins on a selective filtration membrane and to the consequent reduction in time and CAPEX when carrying out the method and obtaining the vesicle of the present disclosure on an industrial scale.

Thus, in a possible implementation form of the first aspect, the liquid composition does not comprise a detergent or a surfactant.

However, by a detergent or a surfactant being included in the liquid composition of the present disclosure, the stability of vesicles in the liquid composition may be improved.

Further, and without wishing to be bound by any particular theory, it is thought that inclusion of a detergent and/or a surfactant in the liquid composition of the present disclosure may lead to increased fluidity of the vesicle membrane, which may encourage the incorporation of a polymer present in the basic solution, such as a reactive end group functionalised PDMS, which may be present in the liquid composition and/or the vesicle and may be functionalized with one or more of amine, carboxylic acid, and/or hydroxy groups, and therefore promote vesicle loading as described herein above.

Thus, in a possible implementation form of the first aspect, the liquid composition may further comprise a detergent or a surfactant. The detergent may be selected from the group consisting of lauryl dimethylamine N-oxide (LDAO), octyl glucoside (OG), dodecyl maltoside (DDM), PEG4000 or combinations thereof. In particular, it is believed that liquid compositions comprising PEG4000 may contribute to the solubility of the vesicles in the liquid aqueous composition.

In a possible implementation form of the first aspect, the plant-derived transmembrane protein and/or the plant plasma membrane components of the vesicle originates from spinach (Spinacia oleracea).

In a possible implementation form of the first aspect the vesicle is for use in a separation membrane.

According to a second aspect, a method of preparing the vesicles in a liquid composition is comprising extracting the plant plasma membrane components and the plant-derived transmembrane proteins from a plant using two-phase partitioning.

In a possible implementation form of the second aspect the mixing the extracted plant-derived transmembrane proteins with a basic formulation.

In a possible implementation form of the second aspect the basic formulation is obtained by dissolving polyethylene glycol-hydroxystearate in phosphate-buffered saline solution.

In a possible implementation form of the second aspect a detergent and/or a flux increasing agent is further added to the basic formulation.

In a possible implementation form of the second aspect the buffer acidity is adjusted to equal to or above pH 7.0, such as equal to or above pH 7.5, such as equal to or above pH 7.8, such as equal to or above pH 8.

In a possible implementation form of the second aspect the total protein concentration of the liquid composition is 5-10 mg/ml.

In a possible implementation form of the second aspect the total water channel transmembrane protein concentration of the liquid composition is between 0.01 μg/ml and 100 μg/ml, such as between 0.1 μg/ml and 10 μg/ml, such as between 0.3 μg/ml and 8 μg/ml.

According to a third aspect, there is provided a separation membrane comprising the vesicle described above.

In a possible implementation form of the third aspect the separation membrane comprises an active layer incorporating the vesicle and a porous support membrane.

In a possible implementation form of the third aspect the active layer comprises the vesicle incorporated in a thin film composite (TFC) layer formed on a porous substrate membrane.

In a possible implementation form of the third aspect, the vesicles are immobilised and/or chemically bound to the TFC layer by naturally occurring free reactive groups, such as amino groups, hydroxyl groups and carboxyl groups, on the surface of the vesicles.

In a possible implementation form of the third aspect the TFC layer is formed by interfacial polymerisation between a di-amine or tri-amine monomer compound and an acyl halide monomer compound.

In a possible implementation form of the third aspect the separation membrane is for use in reverse osmosis membranes.

In a possible implementation form of the third aspect the separation membrane is for use in forward osmosis membranes.

According to a fourth aspect, there is provided a method of preparing a thin film composite layer immobilizing vesicles incorporating a transmembrane protein on a porous substrate membrane, comprising
a. Providing a mixture of vesicles in a liquid composition as disclosed above and a di-amine or tri-amine compound,
b. Covering the surface of a porous support membrane with the mixture of a,
c. Applying a hydrophobic solution comprising an acyl halide compound, and
d. Allowing the aqueous solution and the hydrophobic solution to perform an interfacial polymerization reaction to form the thin film composite layer.

In a possible implementation form of the fourth aspect the di-amine compound is 1,3-diaminobenzene.

In a possible implementation form of the fourth aspect the proportion of the di-amine or tri-amine compound to acyl halide compound is from 0:1 to 30:1 by weight.

In a possible implementation form of the fourth aspect the porous support membrane is formed by a polysulfone or a polyethersulfone polymer.

In a possible implementation form of the fourth aspect the porous support membrane is a flat sheet.

In a possible implementation form of the fourth aspect producing a spiral wound membrane module by winding the flat sheet membrane is further provided.

In a possible implementation form of the fourth aspect the spiral wound membrane module is used for preparing a purified water filtrate by reverse osmosis.

In a possible implementation form of the fourth aspect the spiral wound membrane module is used for the concentration of a product solution by forward osmosis.

These and other aspects will be apparent from and the embodiment (s) described below.

### BRIEF DESCRIPTION OF THE DRAWINGS

FIG. 1 is a bar chart comparison of the water permeability measured in (liters/square meter/hour)/bar, (LMH/bar) and percent salt rejection of E. coli AqpZ containing synthetic polymersome selective membranes (F37) and spinach AQP containing selective membranes (F101) produced in lab scale.

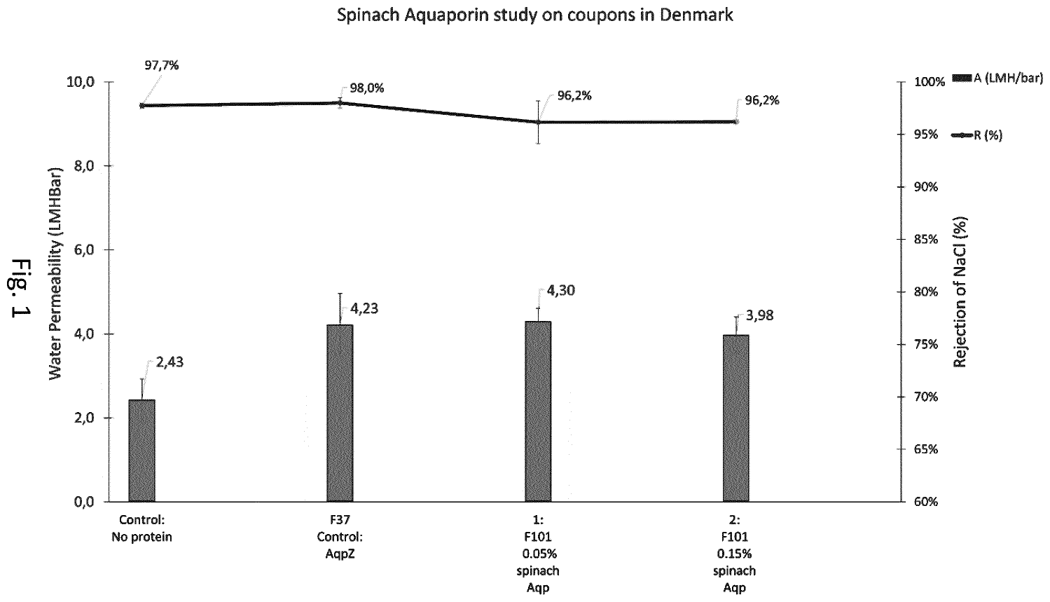

FIG. 2 is a bar chart comparison of the water permeability measured in (liters/square meter/hour)/bar, (LMH/bar) and percent salt rejection of E. coli AqpZ containing synthetic polymersome selective membranes (F37) and spinach AQP containing selective membranes (F101) produced in Pilot industrial scale.

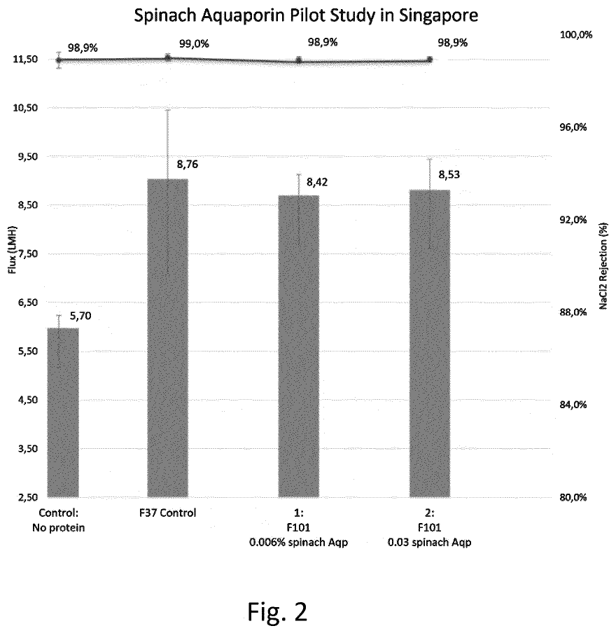

### DETAILED DESCRIPTION

The invention relates to vesicles as disclosed herein, which vesicles comprise plant plasma membrane components and plant-derived transmembrane proteins. The invention further also relates to a separation membrane comprising said vesicles and a method for preparing such vesicles and separation membranes for use in reverse or forward osmosis.

The interfacial polymerization has been extensively described in the art. The reaction of the water phase polyfunctional amide and the organic phase polyfunctional acid chloride produce a polyamide layer on the surface of the support membrane exemplified by the following reaction scheme:

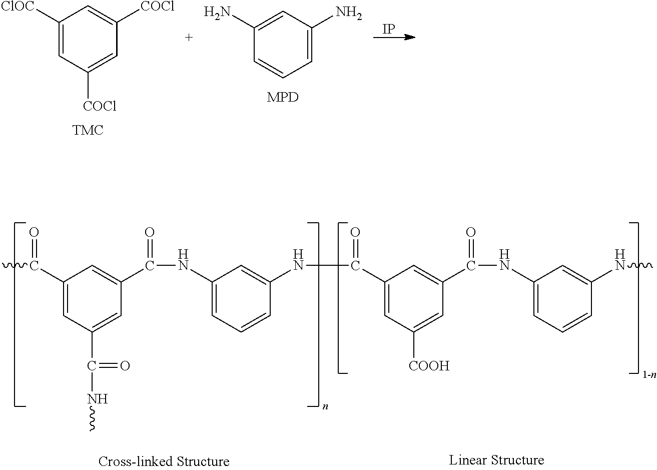

The polyamide layer may comprise a cross-linked structure which influences the permeability of the membrane. A high level of cross-linking results in a tighter membrane structure which results in higher rejection and lower permeability. The formed polyamide layer may comprise a characteristic ridge-and-valley structure which structure may also influence the water permeability of the membrane. The number, size and appearance of the ridges and valleys is dependent on the amount of TMC and MPD used, the ratio of TMC/MPD as well as the additives employed in the water phase and the organic phase. The separation membrane according to the invention may be prepared by adding a liquid composition comprising plant plasma membrane components and plant-derived transmembrane proteins during the membrane fabrication process, such as by adding the liquid composition to an aqueous MPD solution used for forming a TFC layer.

The polyfunctional amine monomer may have primary or secondary amino groups and may be aromatic (e.g., m-phenylenediamine, p-phenyenediamine, 1,3,5-triaminobenzene, 1,3,4-triaminobenzene, 3,5-diaminobenzoic acid, 2,4-diaminotoluene, 2,4-diaminoanisole, and xylylenediamine) or aliphatic (e.g., ethylenediamine, propylenediamine, diethylene triamine, dipropylene triamine, phenylenetriamine, bis(hexamethylene)triamine, bis(hexamethylene)triamine, bis(3-aminopropyl)amine, hexamethylenediamine, N-tallowalkyl dipropylene, 1,3,5-triazine-2,4,6-triamine, and tris(2-diaminoethyl)amine). The polyfunctional amine monomer is suitably a di- or triamine compound. Examples of preferred polyamine species include primary aromatic amines having two or three amino groups, most especially m-phenylene diamine (MPD), and secondary aliphatic amines having two amino groups, most especially piperazine.

The polyfunctional acyl halide monomer is generally a di- or triacyl halide compound, which may be selected among trimesoyl chloride (TMC), trimesoyl bromide, isophthaloyl chloride (IPC), isophthaloyl bromide, terephthaloyl chloride (TPC), terephthaloyl bromide, adipoyl chloride, cyanuric chloride and mixtures of these compounds. The monomeric polyfunctional acyl halide is preferably coated from a non-polar organic solvent, although the polyfunctional acyl halide may be delivered from a vapor phase (for polyacyl halides having sufficient vapor pressure). The polyfunctional acyl halides are preferably aromatic in nature and contain at least two and preferably three acyl halide groups per molecule. Because of their lower cost and greater availability, chlorides are generally preferred over the corresponding bromides or iodides. One preferred polyfunctional acyl is TMC.

The main solvent for the organic phase may be selected from a wide group of compounds. Suitable organic solvents are, for example, one or more of the following non-polar solvents such as hydrocarbons, which may be unsubstituted or substituted. Non-polar solvents include aromatic hydrocarbons, for example mono- or polyalkyl-substituted benzenes, such as toluene, xylenes, mesitylene, ethylbenzene, or mono- or polyalkyl-substituted naphthalenes, such as 1-methylnaphthalene, 2-methylnaphthalene or dimethylnaphthalene, or other benzene-derived aromatic hydrocarbons, such as indane or Tetralin or mixtures thereof. Non-polar solvents also include aliphatic hydrocarbons, for example straight-chain aliphatic compounds of the formula CₙH₂ₙ₊₂, in which n=5-12 or branched aliphatics. Suitable example of straight-chain or branched aliphatic compounds include pentane, hexane, heptane, octane, nonane, decan, undecane, dodecane, 2-methylbutane (iso-pentane), 2,2,4-trimethylpentane (iso-octane), iso-hexane, iso heptane, iso-nonane, iso-dodecane, iso-undecane, iso-dodecane or combinations thereof. Non-polar aliphatic solvents also include cyclic, optionally alkyl-substituted aliphatics, such as cyclohexane or methylcyclopentane; or mixtures thereof.

Non-polar aliphatic solvents are available commercially as the Exxsol® D series, Isopar® series or Bayol® series. A preferred non-polar aliphatic solvent includes Isopar® E, having a distillation range of 115-140° ° C. and an aromatic content of less than 0.002% by weight. Isopar® E mainly comprises C7 to C10 alkanes. Another suitable non-polar aliphatic solvent is Isopar® C having a distillation range of 99-104° C. and a aromatics content of less than 0.001.

Other organic solvents for consideration includes mixtures of aromatic and aliphatic hydrocarbons, such as solvents of the Solvesso® series, for example Solvesso® 100, Solvesso® 150 or Solvesso® 200 Chemicals); (ExxonMobil of the Solvarex®/Solvaro® series (TotalFinaElf); or the Caromax® series, for example Caromax® 28 (Petrochem Carless).

After the polyfunctional amine monomer of the aqueous phase has been allowed to react with the poly functional acyl halide monomer of the organic phase for the formation of a cross-linked polyamide thin composite layer on the support membrane, the solvents and co-solvents are generally allowed to leave the cross-linked polyamide layer by dissolution, rinsing, or washing. In a certain embodiment, the solvents and co-solvents are removed by maintaining the semi-permeable membrane in a bath for a certain time and then allow semi-permeable membrane to dry.

The aquaporin water channels are incorporated in the membrane in the active conformation for at least a significant amount of the molecules. According to an aspect of the invention, the activity of the aquaporin water channels in the vesicle is maintained after incorporation into a membrane, such as TFC layer. Vesicles derived from the plant plasma membrane are the natural environment for the aquaporin water channels, and include naturally occurring phospholipids.

The reactive end group functionalised PDMS (reactive end group functionalized poly(dimethyl siloxane)) which may be present in the liquid composition and/or the vesicle may be functionalized with one or more of amine, carboxylic acid, and/or hydroxy groups. In a certain aspect of the invention the reactive end group functionalised PDMSe-f is bis(amino alkyl), bis(hydroxyalkyl), or bis(carboxylic acid alkyl) terminated PDMSe-f, such as poly(dimethyl siloxane), bis(3-aminopropyl) or poly(dimethyl siloxane), bis(3-hyroxypropyl). Suitably, the integer e is selected in the range of 20 to 40, such as 30 and the integer f is selected from the range of 40 to 80, such as 50. Furthermore, the reactive end group functionalised PDMSe-f may be selected from the group consisting of H2N-PDMS30-50, HOOC-PDMS30-50, and HO-PDMS30-50 and mixtures thereof. When purified aquaporins are added to the liquid composition to optimize aquaporin content of the vesicles, prior to the incorporation of the vesicles with aquaporin water channels, the vesicles may be present in a liquid composition.

The amount of PDMS in any liquid composition is preferably from about 0.05% to about 1% v/v.

Examples of said end-functionalised PDMS are, e.g. bis(aminoalkyl) or bis(hydroxyalkyl) terminated PDMS_{e-f}, where e-f represents the range of from 30 to 50, such as bis(aminopropyl) terminated poly(dimethyl siloxane) having the formula shown here below where (CAS Number 106214-84-0, Aldrich product No. 481246, average Mn ˜5,600 or CAS Number 106214-84-0, product No. 481696 Aldrich, average Mn ˜27,000:

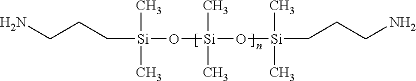

and bis(hydroxyalkyl) terminated poly(dimethyl siloxane) having the formula shown here below where n is approximately 30 to 50 and m and p are both integers between 2 and 5, such as 3 or 4, (CAS Number 156327-07-0, Aldrich product No. 481246, average Mn ˜5,600):

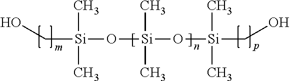

The vesicle and/or the liquid composition of the invention may further comprise a flux improving agent to increase either the water flux or decrease the reverse salt flux. The flux improving agent may be selected among a large group of compounds by is generally preferred as alkylene glycol monoalkyl ether alkylate, beta cyclodextrin, or polyethylene glycol (15)-hydroxy stearate. The flux increasing agent is usually present in an amount of 0.1% to 1% by weight of the liquid composition. The vesicle of the invention may be present in a liquid composition before immobilization in a membrane, such as a TFC layer provided on a support membrane. The liquid composition may comprise a buffer to stabilize the vesicles.

The vesicle and/or the liquid composition of the invention may further comprise a detergent or a surfactant. The detergent may be selected from the group consisting of lauryl dimethylamine N-oxide (LDAO), octyl glucoside (OG), dodecyl maltoside (DDM), PEG4000 or combinations thereof.

The preparation of a thin film composite layer immobilizing vesicles incorporating the aquaporin water channels on a porous substrate membrane comprises providing a mixture of vesicles in a liquid composition prepared as disclosed above, and a di-amine or tri-amine compound, covering the surface of a porous support membrane with the mixture, applying a hydrophobic solution comprising an acyl halide compound, and allowing the aqueous solution and the hydrophobic solution to perform an interfacial polymerization reaction to form the thin film composite layer. In a certain embodiment of the invention, the hydrophobic solution further comprises a TFC layer modifying agent in an amount of 0.1 to 10% by volume. The TFC layer modifying agent has the purpose to increase the water flow and/or the rejection of solutes. In a suitable embodiment, the TFC layer modifying agent is a C3 to C8 carbonyl compound. As an example, the TFC layer modifying agent is selected among the group consisting of diethylene ketone, 2-pentanone, 5-pentanone, and/or cyclopentanone.

The porous support membrane may be a hollow fiber membrane or a flat sheet membrane. Currently, a flat sheet membrane is suitable and may be used for the production of various modules like plate-and-frame modules or spiral-wound modules.

Permeability and rejection are influenced by numerous other factors, such as the choice of support material, monomer concentrations, curing temperature, tension of the support, the pH of the water phase solution, post-treatment of the membrane etc.

Phospholipids are the major components of cell membranes. Phospholipids comprise a hydrophobic triglyceride fatty tail and a hydrophilic head comprising a phosphate group. In biological membranes the phospholipids form a bilayer and typically comprise several other molecules. Several different types of transmembrane proteins span the entirety of the cell membrane to permit or allow the transport of specific substances across the membrane, such as ion channels for e.g. water transport (aquaporins).

It was surprisingly found that plant-derived phospholipid membranes and plant-derived transmembrane proteins may act as suitable water-phase vesicles for TFC membranes in reverse and/or forward osmosis separation membranes or other types of separation membranes. Further, it was found that plant-derived vesicles may be stabilized onto the support membrane so as to withstand the necessary pressure and other environmental conditions when in use.

The porous support membrane may have any physical appearance known in the art, such as flat sheet membrane, tubular membrane, or hollow fiber membrane. In a certain aspect of the invention a hollow fiber membrane is preferred as it provides for higher packing density, i.e., the active membrane area is higher for a certain volume. The membranes may be grouped together or assembled into a module as known in the art. Thus, a plurality of flat sheet membranes may be assembled into a plate-and-frame membrane configuration. Plate-and-frame membrane systems utilize membranes laid on top of a plate-like structure, which in turn is held together by a frame-like support.

Flat sheet membranes may also be assembled into spiral-wound filter modules. In addition to the flat sheet membranes, the spiral-wound membrane modules include feed spacers, and permeate spacers wrapped around a hollow tube called the permeate tube. Spiral wound elements utilize cross flow technology, and because of its construction, can easily be created in different configurations with varying length, diameter, and membrane material. A spiral-wound filter module may be produced by first laying out a membrane and then fold it in half with the membrane facing inward. Feed spacer is then put in between the folded membranes, forming a membrane sandwich. The purpose of the feed spacer is to provide space for water to flow between the membrane surfaces, and to allow for uniform flow between the membrane leaves. Next, the permeate spacer is attached to the permeate tube, and the membrane sandwich prepared earlier is attached to the permeate spacer using glue. The next permeate layer is laid down and sealed with glue, and the whole process is repeated until all of the required permeate spacers have been attached to the membranes. The finished membrane layers then are wrapped around the tube creating the spiral shape.

Tubular membrane modules are tube-like structures with porous walls. Tubular modules work through tangential crossflow and are generally used to process difficult feed streams such as those with high dissolved solids, high suspended solids, and/or oil, grease, or fats. Tubular modules consist of a minimum of two tubes; the inner tube, called the membrane tube, and the outer tube, which is the shell. The feed stream goes across the length of the membrane tube and is filtered out into the outer shell while concentrate collects at the opposite end of the membrane tube.

The hollow fiber membranes may be assembled into a module by assembling a bundle of hollow fibers in a housing, wherein an inlet for passing a first solution is connected to the lumen of the hollow fibers in one end and an outlet is connected to the lumen in the other end, and an inlet is provided in the housing for passing a second solution to an outlet connected to the housing.

The membrane modules produced in accordance with the present invention may be used in various configurations, including forward osmosis configurations and reverse osmosis configurations.

Various plants, such as spinach and their subsequent plant-derived constituents, such as spinach aquaporins are Food Contact Material (FCM) compliant in the EU and the US, since they are regarded safe for use as food ingredients. Spinach comprise a high number of aquaporins in already assembled vesicle structures, called plasma membranes, which structure was found to be retained even after purification.

### Definitions and Terms

The term “transmembrane protein” (TP) as used herein is a type of membrane protein spanning the entirety of the biological membrane to which it is permanently attached in nature. That is, in nature, transmembrane proteins span from one side of a membrane through to the other side of the membrane. Examples of transmembrane proteins are ammonia transporters, urea transporters, chloride channels, and aquaporin water channels.

The term “aquaporin water channel” as used herein includes a functional natural or synthetic aquaporin or aquaglyceroporin water channel, such as aquaporin Z (AqpZ), GlPf, SoPIP2; 1, aquaporin 1 and/or aquaporin 2. Aquaporin water channels include bacterial aquaporins and eukaryotic aquaporins, such as yeast aquaporins, plant aquaporins and mammalian aquaporins, as well as related channel proteins, such as aquaglyceroporins. Examples of aquaporins and aquaglyceroporins include: prokaryotic aquaporins such as AqpZ; mammalian aquaporins, such as Aqp1 and Aqp2; plant plasma intrinsic proteins (PIP), aquaporins, such as tonoplast intrinsic proteins (TIP), nodulin intrinsic proteins (NIP) and small intrinsic proteins (SIP), e.g. SoPIP2; 1, PttPIP2; 5 and PtPIP2; 2; yeast aquaporins, such as AQY1 and AQY2; and aquaglyceroporins, such as GlpF and Yfl054. Aquaporin water channel proteins may be prepared according to the methods described herein or as set out in Karlsson et al. (FEBS Letters 537: 68-72, 2003) or as described in Jensen et al. US 2012/0080377 A1 (e.g. see Example 6).

The terms “separation membrane” as used herein includes membranes useful for separating water and, optionally, certain small size solutes including anions and cations, from other solutes, particles, colloids and macromolecules. Examples separation membranes are “filtration membranes” such as nanofiltration (NF) membranes, forward osmosis (FO) membranes and reverse osmosis (RO) membranes. One type of filtration membranes is a “thin film composite” (or TFC) membrane, often classified as nanofiltration and reverse osmosis membranes. TFC membranes are typically made by depositing a polyamide layer on top of a polyethersulfone or polysulfone porous layer on top of a non-woven or woven fabric support. The polyamide rejection layer is formed through interfacial polymerization of an aqueous solution of an amine with a solution of an acid chloride in an organic solvent. TFC membranes may be produced as described in WO 2013/043118 (Nanyang Technological University & Aquaporin A/S). Other types of filtration membranes are those formed by the layer-by-layer (LbL) deposition method, such as described in Gribova et al. (Chem. Mater., 24: 854-869, 2012) and Wang et al. (Membranes, 5 (3): 369-384, 2015). For example, the self-assembled nanostructure may be embedded or incorporated in the polyelectrolyte multilayer (PEM) films, as outlined in FIG. 4 of Gribova et al.

“Thin-film-composite” or (TFC) membranes as used herein may be prepared using an amine reactant, preferably an aromatic amine, such as a diamine or triamine, e.g., 1,3-diaminobenzene (m-Phenylenediamine, >99%, e.g. as purchased from Sigma-Aldrich) in an aqueous solution, and an acyl halide reactant, such as a di- or triacid chloride, preferably an aromatic acyl halide, e.g. benzene-1,3,5-tricarbonyl chloride (CAS No. 84270-84-8, trimesoyl chloride (TMC), 98%, e.g. as purchased from Sigma-Aldrich) dissolved in an organic solvent where said reactants combine in an interfacial condensation polymerization reaction, cf. Khorshidi et al. (2016) Scientific Reports 6, Article number: 22069, and U.S. Pat. No. 4,277,344 which describes in detail the formation of a composite membrane comprising a polyamide laminated to a porous membrane support, at the surface of the support membrane, e.g. a polyethersulfone membrane. Benzene-1,3,5-tricarbonyl chloride (trimesoyl chloride) is dissolved in a solvent, such as a C6-C12 hydrocarbon including hexane (>99.9%, Fisher Chemicals), heptane, octane, nonane, decane etc. (straight chain or branched hydrocarbons) or other low aromatic hydrocarbon solvent, e.g. Isopar™ G Fluid which is produced from petroleum-based raw materials treated with hydrogen in the presence of a catalyst to produce a low odour fluid the major components of which include isoalkanes. Isopar™ G Fluid: Chemical Name: Hydrocarbons, C10-C12, isoalkanes, <2% aromatics; CAS No: 64742-48-9, chemical name: Naphtha (petroleum), hydrotreated heavy (from ExxonMobil Chemical). Alternatives to the reactant 1,3-diaminobenzene include diamines such as hexamethylenediamine etc., and alternatives to the reactant benzene-1,3,5-tricarbonyl chloride include a diacyl chloride, adipoyl chloride, cyanuric acid etc. as known in the art.

Size of the vesicles: Preferably, the vesicles of the present invention have a particle size of between about 10 nm diameter up to 200 nm diameter depending on the precise components of the vesicles and the conditions used for their formation. It will be clear to those skilled in the art that a particle size refers to a range of sizes and the number quoted herein refers to the average diameter, most commonly mean diameter of that range of particles. The vesicle compositions of the present invention comprise vesicles having mean hydrodynamic diameters of 300 nm or less, in some cases mean diameters that are less than 400 nm such as less than 50 nm.

Forward osmosis (FO) is an osmotic process that uses a selectively-permeable membrane to effect separation of water from dissolved solutes. The driving force for this separation is an osmotic pressure gradient between a solution of high concentration, herein referred to as the draw and a solution of lower concentration, referred to as the feed. The osmotic pressure gradient induces a net flow of water through the membrane into the draw, thus effectively concentrating the feed. The draw solution can consist of a single or multiple simple salt or can be a substance specifically tailored for forward osmosis applications. The feed solution can be a dilute product stream, such as a beverage, a waste stream or seawater, cf. IFOA, http://forwardosmosis.biz/education/what-is-forward-osmosis/Most of the applications of FO, thus fall into three broad categories: product concentration, waste concentration or production of clean water as a bi-product of the concentration when used herein describes process. The term “PAFO” pressure assisted forward osmosis process. The term “PRO” when used herein describes pressure retarded osmosis which is useful in the generation of osmotic power. Membranes of the present invention are useful in all types of forward osmosis processes and may be specifically adapted for each FO type.

The term “reverse osmosis” (RO) is used herein refers to when an applied feed water pressure on a selectively permeable membrane is used to overcome osmotic pressure. Reverse osmosis typically removes many types of dissolved and suspended substances from feed water, including bacteria, and is used in both industrial processes and in the production of potable water. During the RO process, the solute is retained on the pressurized side of the membrane and the pure solvent, the permeate, passes to the other side. Selectivity specifies that the membrane does not allow larger molecules or ions through its pores (holes), while allowing smaller components of the solution (such as solvent molecules) to pass freely. Low pressure reverse osmosis (LPRO) membranes typically operates at a feed water pressure of from about <5 bar and up to a maximum operating pressure of about 25 bar 15 specific flux LMH/bar. LPRO performed at the lower feed pressure ranges, e.g. 2 to 5 bar is sometimes designated ultra-low pressure reverse osmosis. LPRO membranes known in the art have typical operating limits for feed water temperature of about 45° C., feed water pH in the range of 2 to 11, and chemical cleaning in the range of pH 1 to 12.

The present invention is further illustrated with reference to the following non-limiting examples.

### Experimental Section

### Example 1

Production of vesicles comprising aquaporins from spinach leaves.

### Day 1—Preparation of Buffer

1) Buffers and stock solutions. Prepare:
   0.5 M DTT (dithiothreitol) dissolved in miliQ water (stock), store at −20° C.
   0.1 M PMSF (phenylmethylsulfonyl fluoride) dissolved in isopropanol, store at 4° C.
   50 mM EDTA (2,2′,2″,2′″-(Ethane-1,2-diyldinitrilo)tetraacetic acid), pH 8.0 (stock).
   0.2 M KCP (Potassium phosphate buffer), pH 7.8.
   2 M KCl (stock).
   20% Dextran T500 solution in miliQ water (% w/w).
   40% PEG4000 solution in miliQ water (% w/w).
   homogenization buffer: 50 mM MOPS (3-(N-morpholino) propanesulfonic acid), 5 mM EDTA, 0.33 M sucrose, pH 6-7 (adjust with KOH).
   330/5 buffer: 0.33 M sucrose, 5 mM potassium phosphate, pH 7.8.
2) Prepare the aqueous polymer two-phase partitioning system and store at 4° C. (can keep on storage for one month) by the following procedure:
Prepare 3 tubes per purification (270 g for 90 g spinach pellet, derived from 1 kg spinach leaves). Mix gently overnight on a rocker (max tilt, speed 15), 4° C. The sucrose will dissolve during the mixing. Turn the bottles after one hour if needed.
   3.72 g (111.6 g) of 20% Dextran T500 solution
   1.86 g (55.8 g) of 40% PEG4000 solution
   1.08 g (32.4 g) sucrose (will dissolve during mixing)
   225 μL (6.75 mL) of 0.2 M potassium phosphate buffer
   18 μL (540 μL) of 2 M KCL
   Add miliQ water up to 9 g (270 g).
3) Store the glass blender in the fridge overnight, so it is cold for day 2.

### Day 2

### Grinding of Baby Spinach Leaves

Aliquot the needed amount of ascorbate ([Ascorbate]_{final}=5 mM) and PVPP (polyvinylpolypyrrolidon) powder into 3×15 mL falcon tubes, 1 tube per round of homogenization.

Get ice and place a glass beaker with a large funnel with double layered MiraCloth clamped down in the ice to keep cool until use.

1050 grams of organic baby spinach (fresh) was homogenized by 3 rounds blending: ˜350 g of spinach (the stems can be removed, since they contain a lot of lignin) together with 600 mL of homogenization buffer and 1 tube of ascorbate and PVPP.

Blend (homogenize) for 6×15 sec, with 15 sec breaks in between. The “smoothie” should be a fluffy light green mixture without any chunks of spinach left. Use the remaining homogenization buffer to clean the blender.

Filter the “smoothies” through a double layer of MiraCloth into a cold glass beaker (5 L). The liquid should be dark green. The foam will not be filtered, so gently press the remaining liquid through the MiraCloth manually until the pulp is dry. Watch out for breaking the MiraCloth. Discard the pulp.

The final volume is around ˜3 L and now looks like a green juice.

Add 6.7 mL of 0.5 M DTT and 5 mL of 0.2 M PMSF to the 3 L of “green juice” (in the fumehood).

### Differential Centrifugation to Obtain the Microsomal Fraction (MF)

Transfer the 3 L “green juice” to 3×1 L GSA centrifugation tubes (Nalgene) and adjust the weight.

Centrifugation 1: Spin the “green juice” for 30 minutes at 10.000*g, 4° ° C., or for 2 hours at 5300 g, 4° C.

Collect the supernatant and transfer to 2 bottles (˜1.5 L each). The pellet is discarded.

Use a peristaltic pump to collect the supernatant without disturbing the pellet. A plug with holes on top ensures that the surface liquid is being drained. Speed: 170 mL/min.

Aliquot the 3 L “green juice” into 8 smaller (250 mL) GSA the centrifugation tubes (Nalgene), weigh out pairs for centrifuge.

Centrifugation 2: Spin the supernatant for 1 hour and 15 minutes at 25.000*g, 4° C. Discard the supernatant and keep the pellet.

Dissolve the 8× pellets to a total of 90 g in the 330/5 buffer (two phase partitioning buffer) and combine in one tube.

Homogenize the dissolved pellets in the 100 mL Dounce tissue grinders until the solution appear homogenous without clumps (see picture). Add 1 mM DTT and 0.1 mM EDTA to the dissolved pellets. This is the microsomal fraction (MF) and can be frozen down until needed. Otherwise proceed to the first Two-phase partitioning extraction.

### Aqueous Polymer Two-Phase Partitioning to Extract the Plasma Membrane Fractions (PM1 and PM2)

Gently load the 90 g microsomal fraction onto tube 1 of the polymer systems, this becomes upper phase 1 (U1) and lower phase 1 (L1). Load 90 g of clean 330/5 buffer onto tube 2 and 3 of the polymer system, this becomes upper phase 2 and 3 (U2 and U3) and lower phase 2 and 3 (L2 and L3), respectively.

Gently mix tube 1 and 2 overnight on rollers (speed 39 rpm/min., at 4° C.).

Spin for 5 min., 1000 rpm at 4° ° C. to separate the upper and lower phase. Make sure not to mix them upon transport afterwards.

After the first spin:

Transfer the U2 into an empty container. Make sure not to get any lower phase (dextran) with the upper phase (PEG and 330/5 buffer). Transfer U1 onto to L2 gently. Do not transfer any of the L1 and leave the same amount of the U1 as you did of the U2 before.

Transfer U2 back onto to L1.

Gently mix tube 1, 2, and 3 for 1-2 hours on roller (speed 39 rpm/min., at 4° C.). After mixing, check that the weight is approx. paired, before spinning.

Spin for 5 min., 1000 rpm at 4° ° C. to separate the upper and lower phase. Make sure not to mix them upon transport afterwards.

After the second spin:

From tube 3, transfer the U3 into an empty container.

From tube 1, transfer U2 onto to L3 gently. Do not transfer any of the L1 and leave the same amount of the U2 as of the U3 before.

Transfer U3 back onto to L1. This third extraction will not produce any useable plasma membrane fraction from L1. Tube 1 can, however, be used as a counterweight for the 3rd round of centrifugation and discarded be afterwards without ultracentrifugation.

Gently mix tube 1 and 3 for 1-2 hours on roller (speed 39 rpm/min., at 4° C.). After mixing, check that the weight is approx. paired, before spinning.

Spin for 5 min., 1000 rpm at 4° C. to separate the upper and lower phase. Make sure not to mix them upon transport afterwards.

### Ultracentrifugation to Pellet the Plasma Membrane (PM) Fractions

To collect the PM 1 fraction:

Collect U1 from tube 2 without any of L2. It is better to lose a little than to contaminate.

Aliquot 13 mL of U1 into as many Beckmann vials (26.3 mL) as needed. Then add 12 mL of clean 330/5 buffer, so the PEG4000 polymer in the upper phase fraction gets 2-fold diluted. This will enable the precipitation of the plasma membranes during centrifugation.

On the fine balance adjust the weight of the Beckmann vials with lids in pairs (±0.001 g). Use clean 330/5 buffer to adjust the weight.

Spin for 60 min., 100,000*g at 4° C. If the PM fraction have pelleted, then discard the supernatant.

Dissolve/loosen the pellets in 5 mL clean 330/5 buffer and transfer to a 7 mL Dounce Tissue Grinder. Perform ˜10 strokes to homogenize the pellets and get a homogenous mixture without clumps.

Depending on the size of the pellet, additional buffer might be needed to hit a total protein concentration of 5-10 mg/ml. This can be adjusted after total protein determination and will get easier with experience, when you can evaluate on the turbidity of the pellet by eye.

Note the final volume and add 1 mM DTT (2 μL/mL of 0.5 M DTT), 5 mM KCl (25 μL/mL of 2 M KCl), and 0.1 mM EDTA (2 μL/mL of 50 mM EDTA) to the homogenized PM1 fraction. Mix by doing a few strokes.

Aliquot the PM 1 fraction into 1000 μL fractions. Make a 500 μL fraction for QC purposes such as BCA assay. SDS-PAGE, WB, SEC-HPLC and SFLS.

Flash freeze the fractions in liquid nitrogen and store at −80° C.

### To Collect the PM 2 Fraction:

Collect U2 from tube 3 without any of L3.

Aliquot 13 mL of U2 into as many Beckmann vials (26.3 mL) as needed. Then add 12 mL of clean 330/5 buffer, so the PEG4000 polymer in the upper phase fraction gets 2-fold diluted. This will enable the precipitation of the plasma membranes during centrifugation.

On the fine balance adjust the weight of the Beckmann vials with lids in pairs (+0.001 g). Use clean 330/5 buffer to adjust the weight.

Spin for 60 min., 100,000*g at 4° C. If the plasma membranes have pelleted, then discard the supernatant. The PM 2 pellets will be smaller than the PM 1 pellets.

Dissolve/loosen the pellets in 2 mL clean 330/5 buffer and transfer to a 7 mL Dounce Tissue Grinder. Perform ˜10 strokes to homogenize the pellets and get a homogenous mixture without clumps.

Depending on the size of the pellet, additional buffer might be needed to hit a total protein concentration of 5-10 mg/ml. This can be adjusted after total protein determination and will get easier with experience, when you can evaluate on the turbidity of the pellet by eye.

Note the final volume and add 1 mM DTT (2 μL/mL of 0.5 M DTT), 5 mM KCl (25 μL/mL of 2 M KCl), and 0.1 mM EDTA (2 μL/mL of 50 mM EDTA) to the homogenized PM1 fraction.

Mix by doing a few strokes.

Aliquot the PM 2 fraction into 1000 μL fractions. Make a 500 μL fraction for QC purposes such as BCA assay. SDS-PAGE, WB, SEC-HPLC and SFLS.

Flash freeze the fractions in liquid nitrogen and store at −80° C.

### Example 2

### Production of the Support Membrane

A dope was prepared of 17% polysulfone (Solvay P3500 MB7 LCD) dissolved in 83% DMF (N, N-Dimethylformamide) obtained from TACT Chemie. The dope was mixed at a mixing speed of 90 rpm in a closed container at 45° C. for 8 hours for obtaining a uniform viscosity.

The dope was casted on a non-woven polyester sheet (model PMB-SKC) obtained from Mitsubishi in a knife over roll casting mode using a casting gap of 230 μm. After an exposure time 1.9 s a phase inversion was performed by quenching in water at 13° C. for 16 s. Subsequently the support membrane was washed in water at 60° C. for 120 s. A thickness of about 130 μm was obtained.

### Example 3

### 3.1 Production of Basic Formulation

1. Prepare 15 a 0.5% by weight Kolliphor® HS (polyethylene glycol (15)-hydroxystearate) (KHS) solution by dissolving 5 g KHS in 11 PBS (prepared by dissolving 8 g NaCl, 0.2 g KCl, 1.44 g Na2HPO4 and 0.24 g of KH2PO4 in 800 mL MiliQ purified H2O, adjusting the pH to 7.2 with HCl and completing the volume to 1 L).
2. Prepare a 0.5% by weight LDAO (lauryldimethylaminoxide) solution in PBS by dissolving 0.5 g LDAO in 100 mL PBS.
3. Add LDAO 0.05% prepared in step 2 in the proportion 100 mL/L of prepared formulation.
4. Add 0.5% by weight KHS solution prepared in step 1 to reach the desired volume of prepared formulation subtracting the volumes of LDAO.
5. Add amino PDMS to reach a final concentration of 0.1%.
6. Stir the mixture from step 4 overnight at 170 rotations per minute (not more than 20 hours) at room temperature to achieve the basic formulation.
7. Next morning take the prepared formulation obtained in the sequence of steps 1 to 6 and filter it through 200 nm pore size filters to sterilize it, put it in a closed sealed bottle and keep it at room temperature for not more than 12 months.
   3.2 Production of Basic Formulation with 0.15% Spinach Vesicles.

The basic formulation produced in section 3.1 is added 0.15% of the spinach formulation according to example 1.

3.3 Production of Basic Formulation with 0.05% Spinach Vesicles.

The basic formulation produced in section 3.1 is added 0.05% of the spinach formulation according to example 1.

3.4 Production of Basic Formulation with 0.006% Spinach Vesicles.

The basic formulation produced in section 3.1 is added 0.006% of the spinach formulation according to example 1.

### Example 4

### Production of TFC Layer on the Support Membrane

a. Prepare an aqueous solution by mixing in DI water:
   i. 3% MPD
   ii. 3% ε-caprolactam (CAP)
   iii. 5% basic formulation comprising
      0% of the spinach vesicles (example 3.1)
      0.05% of the spinach vesicles (example 3.2)
      0.15% of the spinach vesicles (example 3.3)
b. Prepare an organic solution by mixing in Isopar E:
   i. 0.09% TMC
c. TFC formation
   i. Dipping support membranes according to example 2 in the aqueous solutions for 30 seconds,
   ii. Drying the membrane with air gun at 1 bar,
   iii. Adding the organic solution for 30 seconds,
   iv. Drying the membrane with air gun at about 0.5 bar
d. The membrane with TFC layer was placed in 70° C. 15% citric acid for 4 min and then in 70° C. RO water for 2 min.

TABLE 1
results:

| | A | Rejection |
|---|---|---|
| | LMH/bar | % |
| 0% spinach vesicles | 1.55 | 95.4 |
| 0.05% spinach vesicles | 4.30 | 96.2 |
| 0.15% spinach vesicles | 3.98 | 96.2 |

### Example 5

### Pilot Plant Coating

The experiment of example 4 was reproduced in a larger scale on a pilot plat membrane manufacturing machine for continuous production.

A support membrane roll was placed in an unwind station and the support membrane as produced in example 2 was arranged to travel through a first station for the application of the aqueous phase, and a second station for application of the organic phase. In the first station that membrane is allowed to be in contact with the aqueous phase for 30 sec. The aqueous phase is as prepared in Example 4, however using 0.006% spinach vesicles (example 3.4). Before arriving at the second station, the membrane is dried with an air gun at 1 bar. At the second station, the membrane is contacted with the organic solution for 30 sec. The organic solution is as disclosed in example 4. Finally, the membrane is dried with an air gun at about 0.5 bar.

In addition, the membrane received a post treatment comprising the steps:
1. Contacting the membrane with 10% citric acid of 4 min,
2. Rinsing with water for 2 min,
3. Contacting with 1000 ppm sodium hypochlorite for 2 min,
4. Rinsing with water for 2 min, and
5. Treatment with 1% sodium bisulfite for 1 min.

The membrane was tested for water flux and rejection as above. The result is indicated in table 2 below.

TABLE 2

| | A | Rejection |
|---|---|---|
| | LMH/bar | % |
| 0% spinach vesicles | 7.97 | 98.82 |
| 0.006% spinach vesicles | 8.61 | 98.95 |

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

1. A vesicle in a liquid composition, said vesicle comprising plant-derived transmembrane proteins, wherein the vesicle-forming material comprises plant plasma membrane components.

2. The vesicle according to claim 1, wherein the vesicle forming material consists of plant plasma membrane components.

3. The vesicle according to claim 1, wherein the plant plasma membrane components originate from the same plant as the plant-derived transmembrane proteins.

4. (canceled)

5. (canceled)

6. (canceled)

7. The vesicle according to claim 1, wherein the liquid composition comprises a reactive end group functionalised PDMS (polydimethylsilane).

8. The vesicle according to claim 1, wherein said reactive end group functionalised PDMS is functionalized with one or more of amine, carboxylic acid, and/or hydroxy group(s), and wherein the number of monomers is in the range of 30 to 50.

9. The vesicle according to claim 1, wherein the reactive end group functionalised PDMS is poly(dimethylsiloxane), bis(3-aminopropyl).

10. The vesicle according to claim 1, wherein the plant-derived components comprise plant plasma membrane phospholipids.

11. (canceled)

12. (canceled)

13. (canceled)

14. (canceled)

15. The vesicle according to claim 1, wherein the liquid composition does not comprise a detergent or a surfactant.

16. (canceled)

17. The vesicle according to claim 1 for use in a separation membrane.

18. A method of preparing the vesicles in a liquid composition according to claim 1, comprising extracting the plant plasma membrane components and the plant-derived transmembrane proteins from a plant using ultracentrifugation and/or two-phase partitioning.

19. The method according to claim 18, comprising mixing the extracted plant-derived transmembrane proteins with a basic formulation obtained by dissolving polyethylene glycol-hydroxystearate in phosphate-buffered saline solution.

20. (canceled)

21. (canceled)

22. The method according to claim 18, wherein the total protein concentration of the liquid composition is below 10 mg/ml.

23. The method according to claim 22, wherein the total water channel transmembrane protein concentration of the liquid composition is between 0.01 μg/ml and 100 μg/ml.

24. A separation membrane comprising the vesicle according to claim 1.

25. The separation membrane according to claim 24, wherein the separation membrane comprises an active layer incorporating the vesicle and a porous support membrane.

26. The separation membrane according to claim 25, wherein the active layer comprises the vesicle incorporated in a thin film composite (TFC) layer formed on a porous substrate membrane.

27. The separation membrane according to claim 26, wherein the TFC layer is formed by interfacial polymerisation between a di-amine or tri-amine monomer compound and an acyl halide monomer compound.

28. The separation membrane according to claim 27, wherein the vesicles are immobilized and/or chemically bound to the TFC layer by naturally occurring free reactive groups, such as amino groups, hydroxyl groups and carboxyl groups, on the surface of the vesicles.

29. (canceled)

30. (canceled)

31. A method of preparing a thin film composite layer immobilizing vesicles incorporating a transmembrane protein on a porous substrate membrane, comprising
a. providing a mixture of vesicles in a liquid composition according to any one of the claims 1 to 17 and a di-amine or tri-amine compound,
b. covering the surface of a porous support membrane with the mixture of a,
c. applying a hydrophobic solution comprising an acyl halide compound, and
d. allowing the aqueous solution and the hydrophobic solution to perform an interfacial polymerization reaction to form the thin film composite layer.

32. (canceled)

33. The method according to claim 31, wherein the proportion of the di-amine or tri-amine compound to acyl halide compound is from 0:1 to 30:1 by weight.

34. (canceled)

35. (canceled)

36. (canceled)

37. (canceled)

38. (canceled)
